SEQUENCE LISTING (1 sequence)

SEQ ID NO: 1 - protein, 153 aa, Equus burchelli
GLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

(19) United States
(12) Patent Application Publication
(10) Pub. No.: US 2006/0177887 A1
(43) Pub. Date: Aug. 10, 2006

(54) Method of analyzing a protein

(75) Inventors: Kenji Miyazaki, Tokyo (JP); Hiroaki Torii, Tokyo (JP); Kenichi Kamijo, Tokyo (JP); Akira Tsugita, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: NEC CORPORATION

(21) Appl. No.: 11/322,320

(22) Filed: Jan. 3, 2006

(30) Foreign Application Priority Data
Jan. 4, 2005 (JP) 2005-000216

(51) Int. Cl. C12Q 1/37; G06F 19/00; G01N 33/00

(52) U.S. Cl. 435/23; 436/86; 702/19

(57) ABSTRACT

Provided a method of acquiring information on internal sequence which is applicable to a trace amount of protein and has a high reliability. A protein to be analyzed is limitedly cleaved at a specified position of amino acid to prepare a mixture of a plurality of peptide fragments; one or more peptide fragments are isolated and purified from the peptide fragment mixture; C-terminal amino acids of the isolated and purified peptide fragment are successively released by chemical reaction to prepare a mixture containing a series of resulting products; mass spectrometry is applied to both the reaction products of the successive truncation and unreacted peptide fragment not subjected to the successive truncation reaction; then results of the mass spectrometry is analyzed to acquire chemical structure information of the target protein.

### FIELD OF THE INVENTION

The present invention relates to a technique of analyzing a chemical structure of a protein. More specifically, the present invention concerns a method wherein a protein is cleaved with a protease etc. to produce peptide fragments; a successive truncation reaction is performed from C-terminus of the peptide; molecular weights of reaction products are measured by mass spectrometry; a plurality of partial amino acid sequences including C-terminus of the protein are determined based on measured mass spectra, without use of known information registered in a protein database, as well as a method of analyzing a protein, such as analyzing a kind and position of posttranslational modification and performing a homology search etc, by using obtained amino acid sequence information, and so on.

### BACKGROUND OF THE INVENTION

Methods of analysis for obtaining amino acid sequence information of a protein can be classified into two groups: in methods of one group, experimental results are analyzed by using known database information including amino acid sequences translated and deduced from genomic information; in methods of the other (de novo sequencing), experimental results are analyzed without using known information from such a database.

In Peptide Mass Fingerprinting, widely used in proteomic analysis, for instance, a protein is limitedly cleaved with a protease such as trypsin, the resulting products are subjected to mass spectrometry, then, in reference to known database information, a protein candidate is identified which is cleaved to peptide fragments having mass pattern identical to measured mass pattern. Next, information on an amino acid sequence is obtained indirectly by referring to the amino acid sequence of the identified protein candidate.

However, the number of biological species whose genomic information and protein information are comprehensively registered in a known database is quite restricted. In cases where a comprehensive database is not built, analysis methods cannot be used which depend on sequence information registered in such database. Also, information regarding mature protein such as posttranslational modification and alternative splicing, in general, cannot be obtained only from genomic information. Consequently, de novo sequencing, by which the sequence information of a protein is directly obtained from the protein, is a significant technique for analysis of a protein. As this de novo sequencing, there are two previously known methods.

One is a method based on tandem mass spectrometry (MS/MS), wherein a peptide is fragmented inside a mass spectrometer, and from MS/MS spectrum of the peptide, an amino acid sequence is obtained by mathematical calculation. In this method, an estimated amino acid sequence can be determined by calculation because, if there can be obtained a spectrum in which a series of product ion peaks have been detected, a difference in mass between peaks of the same type of product ions is equivalent to mass of amino acid residue. However, the difficulty in controlling collision energy for cleaving a peptide bond etc. causes, owing to an amino acid sequence, the emergence of easily cleavable position and less cleavable position, the contamination of fragments resulting from concurrent cleavage at two or more positions and so on so that it is difficult to obtain a spectrum in which a series of product ion peaks have been detected. Thus it is not often that the whole sequence of a peptide from the N-terminus through the C-terminus can be read. Moreover, the reliability of the estimated sequence information is not necessarily sufficient.

The other is a method by which sequence information is obtained through the use of chemical reaction. In a representative technique, by using Edman degradation procedure, N-terminal amino acids are successively degraded while resulting amino acid derivatives are identified sequentially. This method, because of use of chemical reaction, can accurately determine a sequence. However, the method has lower analysis sensitivity in comparison with mass spectrometry, and involves disadvantages in analysis speed and running costs etc. On the other hand, as a means to analyze C-terminal amino acid sequence of a protein, there has been proposed a method wherein C-terminal amino acids are successively degraded by chemical technique, and the degraded amino acids are identified from differences in molecular weight between truncated peptides obtained as reaction products thereof and the original peptide. As a means to successively degrade C-terminal amino acids with chemical technique, for instance, there has been proposed a method of allowing vapor generated from a high concentration aqueous solution of pentafluoropropanoic acid or a high concentration aqueous solution of heptafluorobutanoic acid to act on a dried peptide under condition heated up to 90° C., so that selective hydrolysis of the C-terminal amino acid, enhanced by said perfluoroalkanoic acid, is conducted (see Non-Patent Document 1 below). In addition, there has been proposed a method of using, instead of the high concentration aqueous solution of the aforementioned perfluoroalkanoic acid, an acetonitrile solution of pentafluoropropanoic acid anhydride or an acetonitrile solution of heptafluorobutanoic acid anhydride to allow vapor generated from this solution to act on a dried peptide under condition cooled down to, for example, —18° C. so that selective degradation of the C-terminal amino acid, enhanced by said perfluoroalkanoic acid anhydride, is conducted (see e.g. Non-Patent Document 2 below).

It has been reported that, in the aforementioned technique of selectively degrading the C-terminal amino acid by allowing a perfluoroalkanoic acid or perfluoroalkanoic acid anhydride each supplied as vapor to act on a dried peptide, an oxazolone ring structure is formed as a reaction intermediate in a dehydration reaction shown by the following reaction scheme (I): 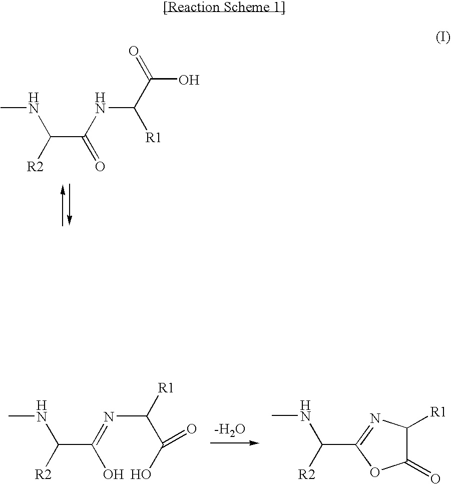
then the perfluoroalkanoic acid acts on this oxazolone ring, resulting in the occurrence of a reaction shown by the following reaction scheme (II): 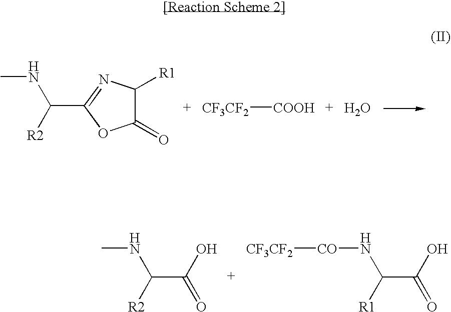
so that a reaction of selective degradation of the C-terminal amino acid is achieved.

The above-mentioned reaction of selectively degrading the C-terminal amino acid proceeds successively. After a predetermined time for the treatment, there is obtained a mixture comprising a series of reaction products each of which is removed one to ten-odd amino acid residues from the C-terminus of the original peptide. This mixture comprising a series of reaction products is subjected to mass spectrometry to measure the mass of the ion species originating from the reaction products, so that there can be obtained a series of peaks exhibiting the mass differences, which reflect the C-terminal amino acid sequences. To be specific, the individual reaction products are formed in reaction of successive releasing C-terminal amino acids from the original peptide so that by applying to mass spectrometry, for example, a series of reaction products of several kinds, each constructed of the original peptides with one to several amino acid residues removed therefrom, the mass of the corresponding ion species can be analyzed collectively, resulting in the determination of C-terminal amino acid sequence of such several amino acid residues at a time.

Moreover, the applicant has been proposed a method of more simple analyzing C-terminal amino acid sequence of a long peptide chain, wherein when C-terminal amino acids of a peptide with its amino acid sequence being long are released successively, in order to inhibit undesired side reactions such as cleavage of a peptide bond at some midpoint inside the peptide, N-acylation pretreatment is applied to a dry sample of the peptide of the long amino acid sequence, and by using a reaction reagent as a combination of an alkanoic acid anhydride and a small amount of a perfluoroalkanoic acid, C-terminal amino acids are degraded in a mild condition and subjected to treatment with added water, followed by lowering, by trypsin digestion, molecular weight of a peptide fragment to be subjected to mass spectrometry (see, for example, Patent documents 1 and 2). This method is useful as an analysis method of great versatile which makes it possible to determine an amino acid sequence consisting of about 10 amino acids from the C-terminus high-accurately and easily. However, this method is still unable to determine the amino acid sequence in an internal region of a protein.

[Patent Document 1]

Japanese Patent Kokai Publication No. JP-P2003-279581A

[Patent Document 2]

JP Patent No. 3534191

[Non-Patent Document 1]

Tsugita, A. et al., Eur. J. Biochem. 206, 691-696 (1992)

[Non-Patent Document 2]

Tsugita, A. et al., Chem. Lett. 1992, 235-238; Takamoto, K. et al., Eur. J. Biochem. 228, 362-372 (1995)

### SUMMARY OF THE DISCLOSURE

A technique to obtain sequence information directly from a protein without reference to known database information is important because the technique makes it feasible to obtain information regarding a mature protein which cannot be obtained only from genomic information. However, the aforementioned Edman degradation procedure and the above-mentioned C-terminal amino acid successive release method can only determine the limited number of amino acid residues from N— or C-terminus so that with such methods it is impossible to determine an amino acid sequence in an internal region of a protein of high molecular weight.

Meanwhile, as a technique for determining an internal sequence of a protein of high molecular weight, there is a technique wherein a protein is limitedly cleaved with trypsin etc., and resulting peptide fragments are isolated and purified by liquid chromatography etc. and then these fragments are subjected to Edman degradation. However, with this method it is impossible to obtain information of high reliability in cases where isolation of such peptide fragments is insufficient. Moreover, in the case of carrying out an analysis to handle a trace amount of protein as a proteomic analysis, it is difficult to isolate and purify peptides of amounts sufficient for application to Edman degradation procedure. Furthermore, in the case of a technique for obtaining an internal sequence using MS/MS techniques, sequence information cannot be often obtained because of difficulty in controlling fragmentation process. Even if sequence information could be obtained, the reliability of analysis result is often insufficient because the reaction mechanism of fragmentation has not fully been clarified. Thus, it is desired to establish an analysis technique which makes it feasible to obtain internal sequence with a degree of high reliability without reference to a known database, and which can be applied even to a trace amount of protein.

The present invention has been made in order to solve the aforementioned problem. The inventors have found out that internal sequence data of high reliability can be obtained promptly and efficiently by that a highly reliable chemical means is employed which is successive release of C-terminal amino acids and a protein is limitedly cleaved in advance at a specified amino acid position with a protease etc.; a peptide mixture, which is made from said limited cleavage products, is isolated and purified by liquid chromatography etc. while said isolated and purified peptides are fractionated and arranged on a plate for mass spectrometric measurement; successive release reaction of C-terminal amino acids is applied to said fractionated and arranged peptide fragments on said plate; successive release reaction products of said C-terminal amino acids are subjected to mass spectrometry; unreacted, isolated and purified peptides are subjected to mass spectrometry; and with respect to mass spectra obtained, comparison and analysis is conducted between a peak originating from unreacted, isolated and purified peptide and a peak originating from a series of reaction products resulting from successive release reaction of C-terminal amino acids. On the basis of these findings, the present invention has been accomplished.

That is, an analysis method of a protein according to the present invention comprises the steps of:
(A) cleaving a protein to be examined limitedly at a specified amino acid position(s) to prepare a mixture of plural peptide fragments;
(B) isolating and purifying one or more peptide fragments from said peptide fragment mixture;
(C) releasing successively C-terminal amino acids of said isolated and purified peptide fragment by chemical reaction to prepare a mixture containing a series of resulting products;
(D) subjecting individually to mass spectrometry both of the products obtained by said successive release reaction and the unreacted peptide fragments not subjected to said successive release reaction;
(E) analyzing results of said mass spectrometry to obtain chemical structure information of the protein to be examined.

It is preferable that in step (B), the means to isolate and purify the peptide fragments comprises fractionating a plurality of fractions by liquid chromatography. Further, it is possible that step (B) comprises a step of dividing said peptide fragment mixture into two fractions, and a step of, with respect to each of said fractions, isolating and purifying one or more peptide fragments, and step (C) comprises a step of successive releasing C-terminal amino acids from the peptide fragments resulting from the step of isolating and purifying one of the divided fraction to prepare a mixture containing a series of resulting products, and a step of maintaining peptide fragments resulting from the step of isolating and purifying the other fraction without subjecting them to the successive release reaction to prepare unreacted peptide fragments. In addition, it is preferable that in the step (C), the chemical reaction is conducted on a plate for mass spectrometric measurement.

In one embodiment, an analysis method of a protein according to the present invention is characterized in that step (C) comprises at least the following steps of:

a pretreatment step of allowing an alkanoic acid anhydride and alkanoic acid both of vapor or droplet phase, which are supplied from a mixture of the alkanoic acid anhydride with a small amount of the alkanoic acid added thereto, to contact with a dry sample on the plate in a dry atmosphere at a temperature selected in a range of 10 to 60° C., thereby protecting the N-terminal amino group of the peptide fragments as well as the amino group on the side chain of the lysine residue which may be included in the peptide by means of N-acylation;

allowing, on the plate, an alkanoic acid anhydride and perfluoroalkanoic acid both of vapor or droplet phase, which are supplied from a mixture of the alkanoic acid anhydride with a small amount of the perfluoroalkanoic acid added thereto, to contact with the dry peptide sample after N-acylation protection in a dry atmosphere at a temperature selected in a range of 15 to 80° C., thereby releasing the C-terminal amino acids at the C-terminus of the peptide accompanied by cleavage of a 5-Oxazolone ring via the formation of a 5-Oxazolone structure represented by the following general formula (III): 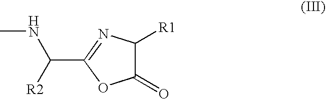
where R1 is a side chain of a C-terminal amino acid of the peptide, and R2 is a side chain of an amino acid residue positioned immediately before this C-terminal amino acid; and

a step of hydrolyzing the C-terminus of the peptide from the reaction products, which comprises applying, to a dried sample containing a series of reaction products obtained in the step of releasing the C-terminal amino acids successively on the plate, a post-treatment of removing the remaining alkanoic acid anhydride and perfluoroalkanoic acid, and then contacting post-treated peptides with a basic nitrogen-containing aromatic compound or a tertiary amine compound and water molecules, all of vapor or droplet phase, using an aqueous solution dissolving a basic nitrogen-containing, aromatic compound or a tertiary amine compound therein.

In a further preferred embodiment, the present method is characterized in that step (C) comprises the following steps, which are repeated at least once:

adsorbing or depositing reaction reagents on the sample by setting the temperature of the plate for mass spectrometric measurement, on which the peptide fragments are spotted and dried, to lower than that of vapor of each reagent used in the steps of the reaction, and

volatilizing the reaction reagents by setting the temperature of the plate to higher than that of vapor of each reagent used in the steps of the reaction.

In a still further embodiment, the analysis method of a protein according to the present invention is characterized in that step (E) comprises the following steps of comparing a mass spectrum of the reaction products obtained by said successive release of the C-terminal amino acids with a mass spectrum of unreacted peptide fragments not subjected to said successive release reaction; identifying a series of amino acids successively released from the C-terminus of said peptide fragment by using the result of said comparison; and arranging said identified amino acids to obtain amino acid sequence information of the protein.

In a preferred embodiment, the step of identifying a series of amino acids successively released from the C-terminus of the peptide fragment consists of

(a) in the mass spectrum of the unreacted peptide fragments,

(a-1) sorting a peak group or groups including isotopic peaks of more than a certain number, which have intensities greater than a certain proportion of an intensity of maximum peak,

(a-2) selecting a single isotopic peak out of said peak group or groups,

(a-3) calculating mass of peptide corresponding to said selected peak and one or more N-acylated forms of said peptide;

(b) in the mass spectrum of a series of the reaction products,

(b-1) searching the presence or absence of a peak which has the mass calculated in step (a-3),

(b-2) in case where the peak is present, choosing the peak as a candidate peak of a peptide fragment not truncated from the C-terminus or an N-acylated form thereof,

(b-3) calculating a value by subtracting, from mass of the candidate peak of the N-acylated form, mass of the endmost amino acid residue of the peptide produced by the limited cleavage

(c) in the mass spectrum of a series of the reaction products,

(c-1) searching the presence or absence of a peak which has the mass of the value calculated in step (b-3),

(c-2) in case where said peak is present, identifying the peak as a peak obtained by elimination of one C-terminal amino acid from the peptide fragment;

(d-1) identifying a peak group corresponding to a series of reaction products resulting from successive dissociation of C-terminal amino acids relative to said identified peak to be used as a reference (a base point), and based on the peak group, calculating reduction of molecular weight which is caused by successive release of amino acids from the C-terminus, and then

(d-2) identifying a series of amino acids resulting from successive release based on a series of calculated reduction of molecular weight.

In a further more preferred embodiment, the present method is characterized in that in the mass spectrum of a series of the reaction products, in case where a candidate peak of the peptide fragment not truncated from the C-terminus, as identified in step (b-2), or the N-acylated form thereof is present, and a peak of the value calculated in step (b-3) is absent, it is judged that there is a possibility that the peptide fragment not truncated from the C-terminus is a C-terminal peptide fragment of a protein to be analyzed; there is identified a peak group corresponding to a series of reaction products resulting from successive release of C-terminal amino acids relative to said judged peak to be used as a reference (a base point); based on said peak group, reduction of molecular weight is calculated which is caused by successive release of amino acids from C-terminus; and based on a series of the calculated reduction of molecular weight, a sequence of successively released amino acids is identified to determine the identified amino acid sequence as a C-terminal amino acid sequence of a protein to be analyzed.

It is possible that in cases where genetic information of the protein to be analyzed is known, without reference to the mass spectrum of said unreacted peptide fragment, there is obtained information as to chemical structure of a protein to be analyzed, based on the mass spectrum of the reaction products obtained by successive release of said C-terminal amino acids. On the other hand, it is preferred that, in cases where a protein having unknown amino acid sequence information is analyzed, there is made a precise measurement involving addition of internal standard peptides, in conducting mass spectrometry of said unreacted peptide fragment.

In another aspect, the present invention provides a method of identifying a homologous protein comprising the steps of determining a plurality of partial amino acid sequences of a protein to be analyzed with use of any one of the aforementioned methods; and implementing, by using the determined plural partial amino acid sequences, homology search for proteins registered in a known protein database to identify a protein homologous to a protein to be analyzed.

In a still another aspect, the present invention provides a method of analyzing a posttranslational modification of a protein. The method comprises the steps of, with respect to a protein to be analyzed having a known amino acid sequence, determining, by using any one of the aforementioned methods, mass numbers and partial amino acid sequences of a plurality of peptide fragments originating from the protein to be analyzed; calculating, based on said known amino acid sequence, theoretical mass numbers of peptide fragments produced when the protein to be analyzed is subjected to a hypothetical limited cleavage at a specified amino acid position(s); identifying the positions of the peptide fragments on the protein to be analyzed based on the partial amino acid sequences; comparing, with respect to the peptide fragments with identified position, the mass measured by mass spectrometry with said theoretical mass; judging, in cases where there is a difference between the measured mass and the theoretical mass, that there is a possibility that a modifying group is present in the position-identified peptide fragment; estimating, with respect to the peptide fragment judged that it has a possibility of the presence of the modification, a kind of the modifying group based on the difference between the theoretical mass and the measured mass; and judging a position of an amino acid modified with the estimated modifying group from a position at which there disappears the difference of mass calculated from the spectrum measured in association with the successive release. This analysis method can also be applied to a protein having an unknown amino acid sequence by, in advance, searching a known database with use of precise mass of a peptide and its partial amino acid sequence to identify a protein to be analyzed.

The meritorious effects of the present invention are summarized as follows.

The present method makes it possible to obtain highly reliable internal sequence information of a protein without use of information of database. Further, in the present method, the employment of highly reliable chemical technique, i.e. successive release of C-terminal amino acids of a peptide, enables acquisition of highly reliable internal sequence concerning also a protein sample which was difficult to analyze its internal sequence by MS/MS analysis. Moreover, the employment of the original procedure for an analysis method of a mass spectrum enables to obtain highly reliable internal sequence information also on a protein sample which was difficult to analyze by conventional Edman degradation owing to the insufficiency of isolation of peptide fragments by liquid chromatography. Also, by analysis with use of mass spectrometry, a trace amount of sample can be analyzed, too, which was difficult to analyze by a method with use of conventional Edman degradation.

### BRIEF DESCRIPTION OF THE DRAWINGS

FIG. 1 shows a flow chart of steps for determination of amino acid sequence of a protein according to one embodiment of the present invention

FIG. 2 shows a flow chart of steps showing an analysis method of mass spectrum according to one embodiment of the present invention

FIG. 3 shows Mass spectrum of peptide fragment (without successive release reaction of C-terminal amino acids) originating from one fraction isolated by Liquid chromatography

FIG. 4 shows Mass spectrum of a series of products obtained by conducting successive release reaction of C-terminal amino acids on a plate for mass spectrometric measurement with respect to peptide fragment originating from one fraction isolated by Liquid chromatography

FIG. 5 shows an enlarged picture of a partial region of mass spectrum shown in FIG. 4

FIGS. 6A and 6B show schematic diagrams in which it is analyzed by a method of the present invention whether posttranslational modification of a protein is present or not.

### PREFERRED EMBODIMENTS OF THE INVENTION

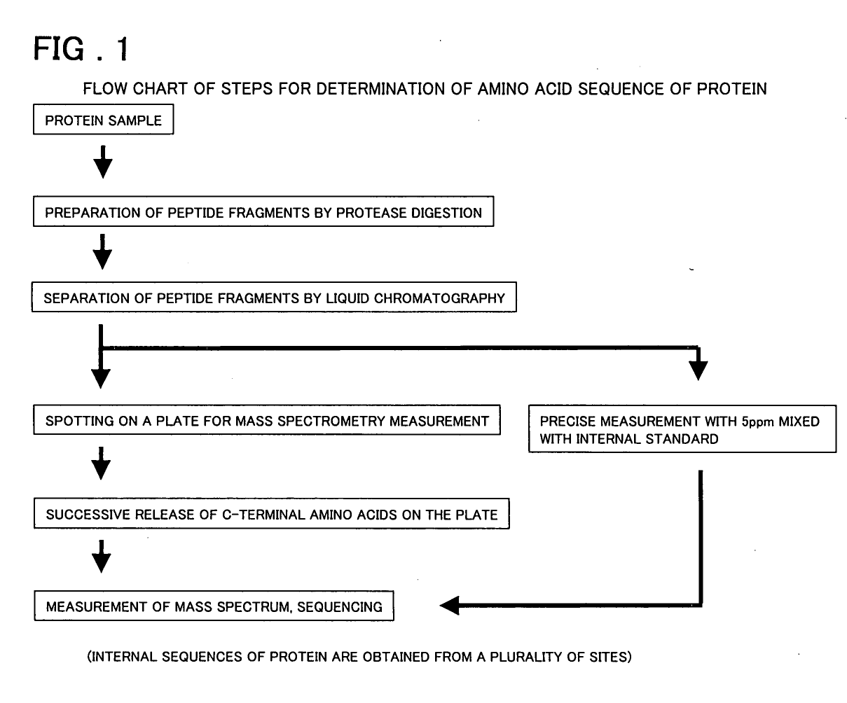

One embodiment of a protein analysis method of the present invention is described by using a flow sheet shown in FIG. 1. In a method of the present invention, a plurality of peptide fragments are first prepared by the limited cleavage of a protein to be analyzed at a specified amino acid position(s) with a protease etc previously, for the purpose of acquiring not only amino acid sequences from the C-terminus of a protein, but also internal amino acid sequences. Next, the above-mentioned peptide fragments are separated to one or more fractions by liquid chromatography. A part of the obtained peptide fragments of a fraction is dropped and dried on a plate for mass spectrometric measurement, then on this plate, C-terminal amino acids thereof are successively released and eliminated by chemical means to prepare a series of reaction products with their limitedly truncated peptide chains. Regarding both a sample containing these successive release reaction products of C-terminal amino acids and unreacted peptide fragment fractionated by liquid chromatography, molecular weights thereof are measured by mass spectrometry; from mass spectra obtained, the reduction of molecular weight is calculated which has been caused by successive release of amino acids; based on a series of molecular weight reductions calculated, a series of amino acids subjected to successive release are identified to obtain information on a amino acid sequence. In this case, it is preferred that in mass spectrometry of unreacted peptide fragment, there is performed a precise measurement wherein a sample, mixed with internal standard peptides, is subjected to mass spectrometry. These steps are explained in detail below one after another.

(Preparation of Peptide Fragment Mixture by Limited Cleavage of Protein)

Proteins to be analyzed by the present invention are not limited to particular ones, but may be any proteins occurring naturally or any proteins synthesized artificially. Proteins being present in nature may derive from any biological species. Amino acids composing a protein may include, in addition to twenty kinds of amino acids existing in nature, optical isomers thereof and other nonnatural amino acids.

In a method of the present invention, a protein to be analyzed is first cleaved limitedly at a specified position(s) to prepare a plurality of peptide fragments. The method of limited cleavage used herein may be any methods, regardless of their concrete means, that make it possible to cleave a protein at specific amino acid residue, preferable methods being, for example, a chemical method with use of cyanogen bromide which cleaves a protein specifically at a carboxyl side of methionine residue therein and a method with use of an enzyme which carries out cleavage at a carboxyl group side of a specified amino acid residue. As such enzymes, there can be used, for example, trypsin cleaving a protein at a carboxyl side of lysine or arginine residue therein, animal-derived digestive enzymes such as chymotrypsin specific to a large hydrophobic side chain contained in phenylalanine, tyrosine, tryptophan and so on. In addition, a variety of microorganism-derived proteases can be also used, including, for example, protease V8 which is prepared from Staphylocossus aureus V8 cell line and specifically cleaves a peptide bond at a carboxyl side of aspartic acid or glutamic acid. (It is possible to cleave only at a carboxyl group side of glutamic acid by selecting reaction conditions.)

(Isolation and Purification of Peptide Fragments)

Subsequently, one or more peptide fragments are subjected to isolation and purification from a peptide fragment mixture prepared by the aforementioned method. The isolation and purification method may be any methods known to a person of ordinary skill in the art, and can be conducted by SDS-polyacrylamide gel electrophoresis (SDS-PAGE) or capillary electrophoresis, or a variety of chromatography. In preferred embodiments, the above-mentioned isolation and purification means is performed by fractionation of a plurality of fractions by means of liquid chromatography. As used in a method of the present invention, “liquid chromatography” refers to a separation technique by chromatography using liquid as a mobile phase, which is utilized for several kinds of applications as high performance liquid chromatography (HPLC) operable under pressure by suitably selecting a stationary phase column. Separation mechanisms can be classified under various kinds of mechanisms such as partition, adsorption, ion exchange, size elimination etc. A most frequently used column has a reverse phase stationary phase, separation being performed by partition, a sample being retained by stationary phase of lower polarity and eluted by mobile phase of polarity. For example, a separation column can be used which has, as a separation carrier, silicide with ODS (octadecylsilyl group).

Recently, there has been developed chromatography and fractionation system for supplying many samples in suitable conditions to analysis system such as electrospray ionization or MALDI-TOF/MS employed in mass spectrometry, i.e. for high-throughput analysis. They include Micro liquid chromatography (Micro LC) with flow rate of not greater than 1000 μl/min or Nano liquid chromatography (Nano LC) with flow rate of not greater than 1000 nl/min, which can be subjected to a method of the present invention. In particular, Nano LC, which enables punctate application (spotting) of a trace amount of samples and a minute quantity, can provide a sample appropriate for a sample for mass spectrometry of the present invention.

(Successive Release Reaction of C-terminal Amino Acids)

A peptide fragment contained in each of the fractions which have been fractionated by the aforementioned liquid chromatography is dropped onto a plate for mass spectrometric measurement, dried to prepare a mixture containing a series of products obtained by successive releasing C-terminal amino acids of the peptide fragment on the plate. As used herein, successive release reaction of C-terminal amino acids refers to a method wherein N-acylation treatment is applied in advance to a dry sample of a peptide to split C-terminal amino acids away under mild conditions by using a reaction reagent in combination of alkanoic acid anhydride and a small amount of perfluoroalkanoic acid. This method has been published in relation to the patent application of the present applicant (see the above-mentioned Patent documents 1 and 2), the contents of which are incorporated herein by reference. The invention of the present application is characterized in that this successive release method of C-terminal amino acids is carried out on a plate for mass spectrometric measurement, that is, applied to each peptide derived from the isolated and purified protein isolated and purified by e.g. liquid chromatography. As used herein, “a plate for mass spectrometric measurement” refers to a plate for spotting of a sample containing peptide fragments in order to implement measurement by MALDI-TOF-MS method. The plates used herein may be any plates for mass spectrometric measurement by MALDI-TOF-MS method, and preferably include a plate protected with material resistant to acidic or basic reagent used in successive release reaction of C-terminal amino acids, for instance with platinum- or gold-plating.

In order to efficiently conduct successive release reaction of C-terminal amino acids on this plate, it is required to supply a sufficient quantity of reaction reagent. However, too excessive amount of reagent causes condensation on the plate, whereby contamination of plural specimens on the plate occurs possibly, resulting in the difficulty of analyzing measurement results by MALDI-TOF-MS. It is therefore required to strictly control condition for supply of reaction reagent such that a plurality of samples are not contaminated. In an exemplary method, reaction reagent, as a liquid drop, is supplied to a sample under direct control of the drop.

Preferably, in successive release reaction of C-terminal amino acids, adjustment of a temperature of a plate for mass spectrometric measurement on which dried samples of the peptide fragments are fractionated and arranged, and a temperature of vapor or droplet of the above-stated alkanoic acid anhydride and perfluoroalkanoic acid enables promotion of adsorption (vapor deposition) and volatilization, resulting in increase of reaction efficiency. In order to facilitate adsorption of reagent, a temperature of the plate is set to a temperature being by 1 to 10° C., preferably by about 5° C. lower than that of the vapor or droplet of the reagent because in such condition, the heat energy of the vapor of the reagent is removed on the surface of the plate to become easy to liquefy. Meanwhile, if a temperature of the plate is set to a temperature being by 1 to 10° C., preferably by about 5° C. higher than that of the vapor or droplet of the reagent, it is possible to promptly volatile the remaining reagent after completion of the reaction. The temperature of a plate can be easily adjusted by, for example, a temperature adjustment mechanism with use of known Peltier device etc.

(Measurement and Analysis of Mass Spectrum)

By using a mixture containing a series of products obtained by the aforementioned method, analysis by mass spectrometry is conducted. Particularly, it is preferred that measurement of mass spectrum is performed by MALDI-TOF-MS method. In this method, a peptide sample is mixed with crystalline matrix (a-cyano-4-hydroxycinnamic acid, for example). This crystalline matrix plays a role in dispersing peptides, absorbing laser light, and causing a transition of the energy thereof to analysis molecules (peptides). The peptide is ionized by receiving a proton from an optically pumped crystalline matrix, typically, to generate ionic chemical species of (M+H)⁺ type. Through this soft laser desorption process, transition of peptides to gas phase occurs. A peptide is ionized, so the ion is accelerated in an electric field, detected with a detector, and the time between ionization and detection is measured and calculated highly accurately. The mass can be accurately calculated because of dependency of flight time of an ion on momentum and the square root of mass to charge ratio (m/z).

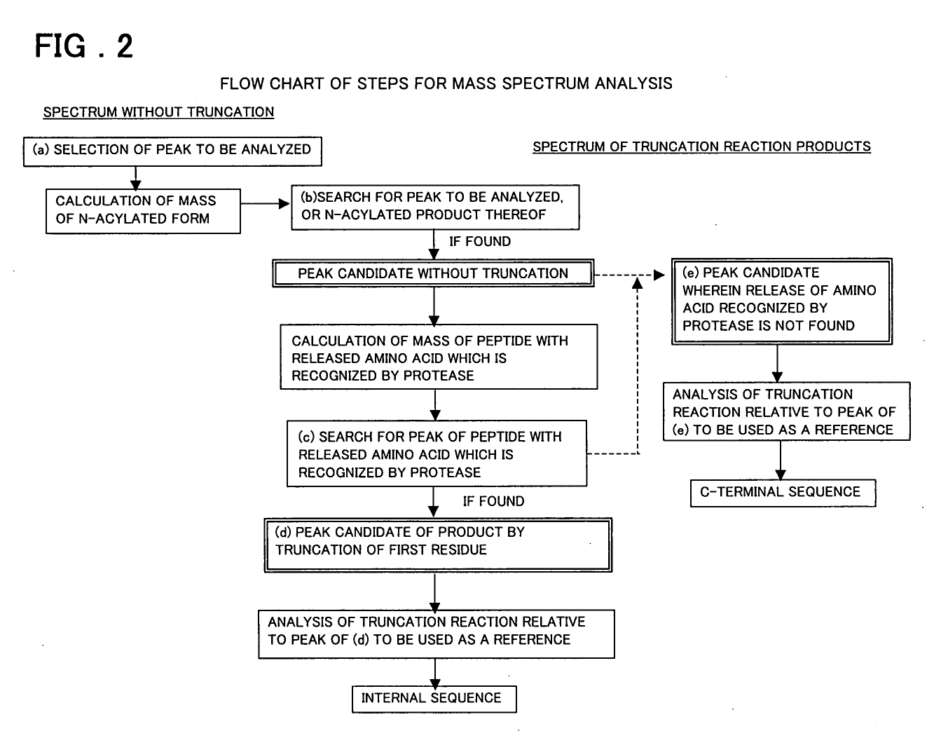

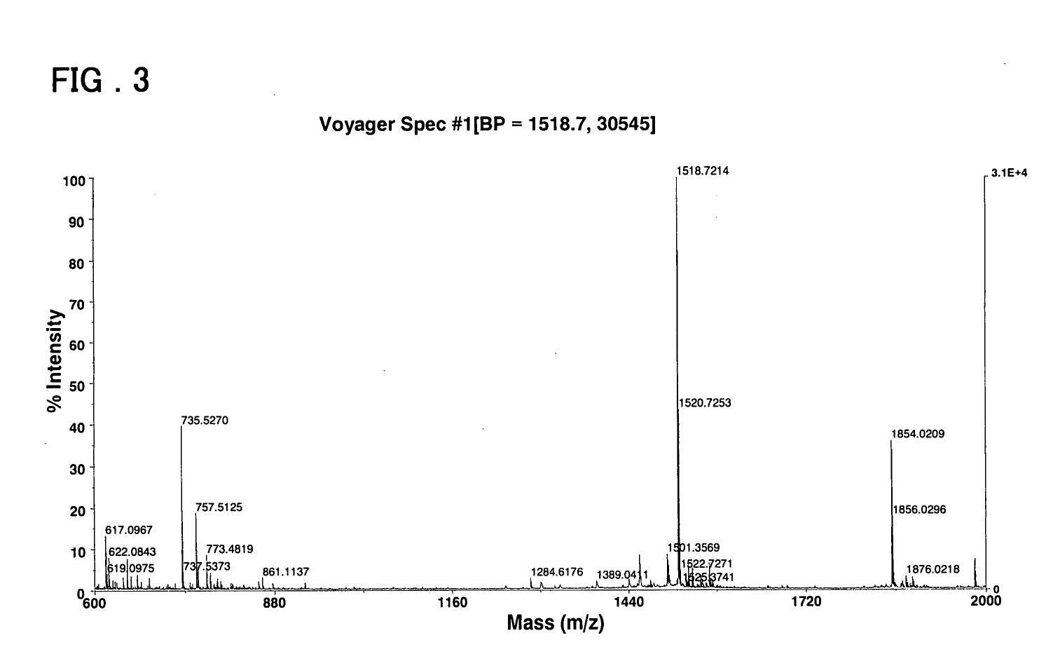

The mass spectra obtained in this manner are subjected to analysis by a method exemplarily shown in FIG. 2. Initially, in the first step (a), analysis is applied to a spectrum of unreacted peptide fragment(s) contained in one fraction that has been fractionated by liquid chromatography. The resulting spectrum corresponds to, for example, a spectrum as shown in FIG. 3. Such fraction may contain a plurality of peptide fragments so it is required to sort peaks originating from one or more peptides. In this sorting process, a peak group is sorted which preferably has not less than 5% in intensity, relative to a peak of maximum intensity, and involves three or more isotope peaks, and a single isotope peak is selected from among such peak group. The selected peak is determined as an object to be analyzed. The presence of isotope peak with appropriate intensity ratio suggests that the peak is the one originating from a natural peptide. Natural proteins contain several kinds of stable isotopes. It is known that for instance, even though abundance ratio of ¹³C is circa 1%, one or more isotope peaks are observed by mass spectrometry in the case of polymeric compounds such as proteins or peptides. Thus there is a high possibility that a peak group having proper intensity ratio and involving three or more isotope peaks is the one originating from a peptide fragment. Hence by sorting a peak group involving not less than three peaks and then selecting a single isotope peak from among the peak group, a peptide-originating peak can be selected with a high degree of accuracy.

Once a peak to be analyzed is selected, there is then calculated mass of the peptide corresponding to the peak and mass of N-acylated body of the peptide. This acylating group gives protection to the amino group of the N-terminus of a peptide and the amino group of the side chain of lysine residue possibly contained in the peptide. N-acylating- and O-acylating reactions proceed concurrently owing to use of a combination of alkanoic acid anhydride being an electrophilic acylating agent, as a reaction reagent, and alkanoic acid, as a catalyst intended to promote the acylating reaction, by its ability of proton donation. Concretely, there are utilized alkanoic acid having 2 to 4 carbon atoms and symmetric acid anhydride originating from such alkanoic acid having 2 to 4 carbon atoms. By using molecular weight of N-acyl group corresponding to these reagents, calculation of mass of one or more N-acylated body is carried out. In general, a combination of acid anhydride and acetic acid is used so that there is performed addition of mass corresponding to molecular weight (42.04 Da) of one or more acetyl group.

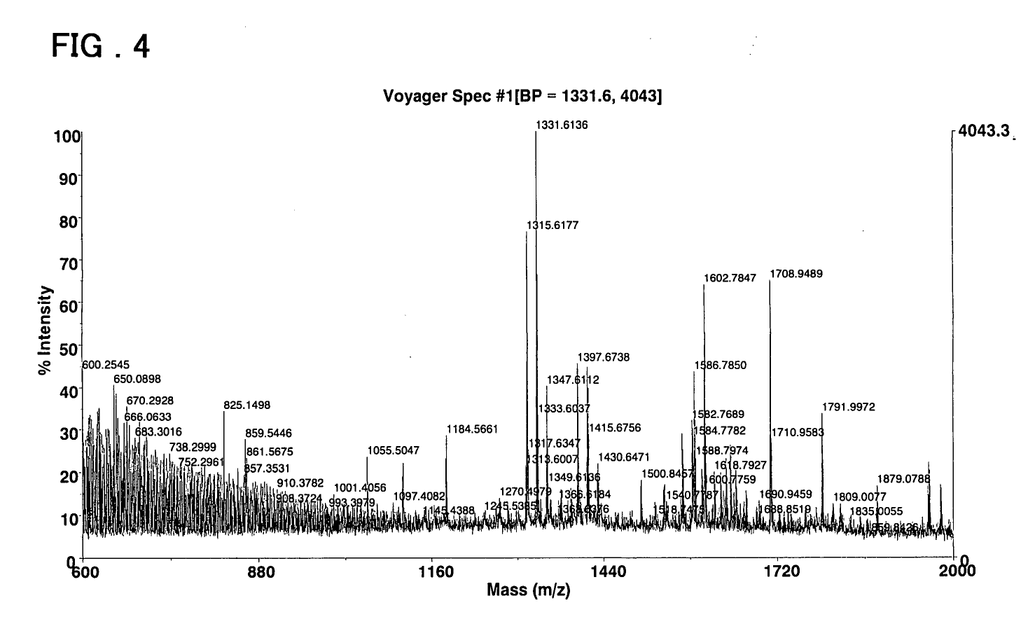

In the next step (b), there is analyzed mass spectrum (shown in FIG. 4, for example) of a series of reaction products obtained from successive release of C-terminal amino acids of the aforementioned peptide fragments. First, it is searched whether a peak of the N-acylated peptide fragment that has been calculated in the step (a) as mentioned above is present or not. As a result, if the peak is found out, such peak is chosen as a candidate peak of a peptide not subjected to degradation of C-terminal amino acids. If it is not found out, it is searched whether a peak having mass identical to that of the peak selected in the step (a) is present or not. As a result, if it is found out, such peak is chosen as a candidate peak of a protein not subjected to degradation of C-terminal amino acids. Then, a value is calculated by subtracting, from the mass of this peak candidate, the mass of amino acid residue recognized by the above protease. This value corresponds to the mass of a peptide with one C-terminal amino acid released from the peptide of the aforesaid chosen peak candidate.

In the next step (c), there is analyzed mass spectrum of a series of reaction products resulting from successive releasing C-terminal amino acids of the above-mentioned peptide fragment similarly to search whether a peak of mass that has been calculated in the step (b) is present or not. If it is found out, such peak is identified as a peak obtained by removal of one C-terminal amino acid from the above-stated peptide fragment. With respect to the identified peak to be used as a reference (a base point), there is identified a peak group corresponding to a series of reaction products resulting from dissociating C-terminal amino acids in sequence. Based on the peak group, the reduction of molecular weight associated with successive release of amino acids from C-terminus can be calculated (step (d)). Meanwhile, in the case of the absence of a peak of the mass number that has been calculated in the step (b), there is a high possibility that the aforesaid chosen candidate peak is the C-terminal peptide of a protein. With the identified peak being as a base point, there is identified, by a method similar to the above-mentioned one, a peak group corresponding to a series of reaction products resulting from dissociating C-terminal amino acids in sequence. Based on the peak group, the reduction of molecular weight accompanied by successive release of amino acids from C-terminus can be calculated (step (e)).

Based on a series of the thus measured reduction amounts of molecular weight of one or more peptide fragments, a series of amino acids that have been successively released are identified, and the identified amino acids are arranged to obtain chemical structure information such as information on amino acid sequence of a protein to be analyzed. As used herein, “chemical structure information” basically includes, but is not limited to, information as to the aforementioned amino acid sequence. In the present invention, it is possible to analyze, as explained below in detail, posttranslational modification of a protein, splicing variants, genetic variant proteins, and a regulation mechanism of proteolysis such as ubiquitylation, the information thereon being also included in chemical structure information on a protein of the present invention.

For instance, the above-mentioned analysis method, in cases where it is performed by means of two or more proteases with substrate specificities different from each other, makes it feasible to determine the whole amino acid sequence of a protein. Further, by identifying a peptide fragment (a peptide fragment containing, at its C-terminus, a amino acid(s) other than lysine or arginine, for example, in the case of trypsin cleavage) not containing a amino acid(s) that should be present at the C-terminus of a peptide fragment degraded by a used limited cleavage technique, i.e. based on the specificity of a used protease, it is possible to determine the C-terminal amino acid sequence of a protein to be analyzed.

An analysis method of a protein according to the present invention can be suitably applied to both proteins with known and unknown amino acid sequences. In cases where genetic information on a protein to be analyzed is known, for example, an analysis method of the present invention can be easily carried out with use of amino acid sequence information assumed from such genetic information. That is, in this case, without reference to the mass spectrum of the unreacted peptide fragment, chemical structure information regarding a protein to be analyzed can be obtained by comparing the mass spectrum of the peptide that has been subjected to successive release of amino acids from C-terminus with amino acid sequence information thereon. On the other hand, where a protein with unknown amino acid sequence information is to be analyzed, a precise measurement is preferably conducted wherein internal standard peptide(s) is added in mass spectrometry of the unreacted peptide fragment. As internal standard peptide, angiotensin I or II etc. can be utilized.

In another aspect, the present invention provides a method of identifying a protein homologous to a protein to be analyzed, wherein a plurality of partial amino acid sequences of a protein to be analyzed are determined by using any one of the above-mentioned methods, and homology search for proteins registered in a known protein database is implemented by using the determined plural partial amino acid sequences. The degree of homology of proteins can be represented as a percentage of identity when carrying out alignment between the amino acid sequences of two proteins appropriately, and means an incidence rate of accurate concordance between these sequences. Appropriate alignment between sequences for identity comparison can be determined by using a variety of algorithm such as BLAST algorithm (Altschul SF J Mol Biol October 5 1990; 215(3): 403-10).

(Analysis of Posttranslational Modification of Protein)

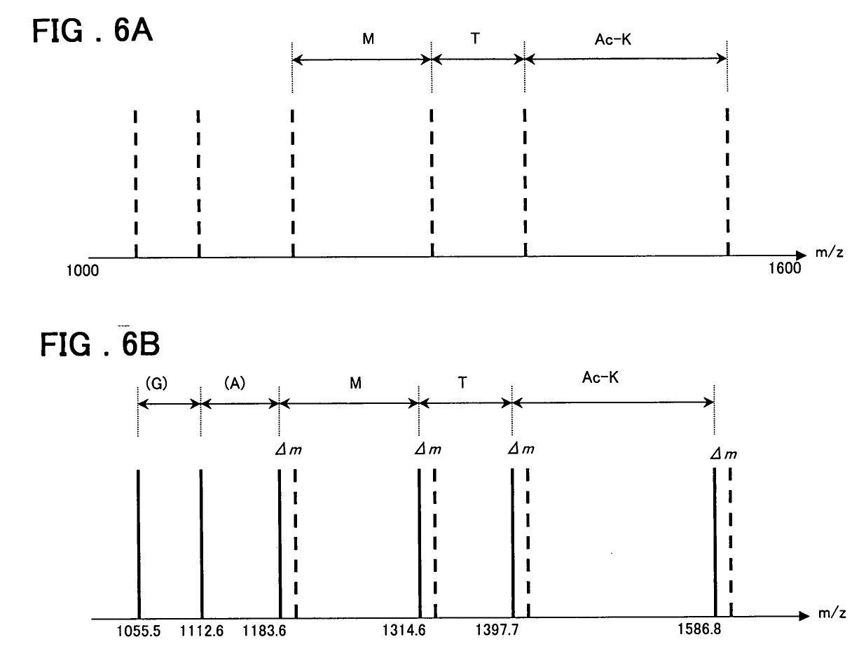

A method of the present invention may be also applicable to an analysis of posttranslational modification of a protein. For example, FIGS. 6A and 6B show schematic diagrams to explain such analysis method. A mass spectrum resulting from analysis of a sample protein by an analyzing method of the present invention is indicated in FIG. 6A. Hereof it is considered that as a result of analysis of this mass spectrum, for example, there have been able to be identified, with respect to a lysine residue at C-terminus to be used as a reference (a start point), four peaks corresponding to molecular weight of threonine and methionine in sequence.

Furthermore, it is considered that this protein has been identified as myoglobin from information on a plurality of peptides other than the peptide as mentioned above. So, by matching of the positions concerned of partial sequence MTK, it is concluded that this peptide is a part of peptide of [119-133]. Mass of products by successive release of C-terminal amino acids of this peptide is predicted as follows:

| | |
|---|---|
| [119-133]: HPGNFGADAQGAMTK | 1586.8 Da |
| [119-132]: HPGNFGADAQGAMT | 1397.7 Da |
| [119-131]: HPGNFGADAQGAM | 1314.6 Da |
| [119-130]: HPGNFGADAQGA | 1183.6 Da |
| [119-129]: HPGNFGADAQG | 1112.6 Da |
| [119-128]: HPGNFGADAQ | 1055.5 Da |

Here it is considered that the mass numbers of the above-identified four peaks do not agreed with the above predicted mass numbers, and evenly shifted by Δm on an m/z axis. In this case, if superposition of the aforementioned spectrum and predicted spectrum appears as is shown in FIG. 6B, three peaks on a heavy-mass side are shifted by Δm from their respective peaks predicted theoretically. This results in a conclusion that a modification with mass change of Δm is present. Meanwhile, two peaks whose relevant amino acids have not been able to be identified in FIG. 6A coincide with their correlated peaks predicted theoretically. This indicates that there exists no modification in a peptide corresponding to two peaks on a low-molecular weight side, i.e. the position of the modifying residue is a C-terminal amino acid (in this case, alanine (A)) of a peptide relating to a peak having minimum m/z among four peaks showing an increase in Δm.

Moreover, for example in the case of Δm being equal to 14 [Da], there is concluded a possibility that methylation (one of known modifications) to this peptide has been caused. Also, the result shows that it is alanine to which the modification has been caused because in the above description, the mass of the peptides after removal of alanine each agrees with their respective theoretical values. Therefore, there is concluded a possibility of methylation to alanine in the above example. It is thus possible to estimate, with respect to known modification, the presence thereof and the position of a modifying residue.

The aforementioned inference framework is also useful in the case of a type of modification corresponding to Δm being unknown. In this case, there is only a possibility that, to a modified amino acid, some type of modification involving mass change of Δm has occurred. It is thus possible to estimate, with respect to unknown modification, the presence thereof and the position of a modifying residue. Likewise, by detecting mass change, an analysis of splicing variants, genetic variant proteins, and a regulation mechanism of proteolysis such as ubiquitylation can be also carried out.

In a method according to the present invention, performing the successive release reaction of C-terminal amino acids on a plate for mass spectrometry measurement makes it feasible to parallel treat a large number of objects, resulting in high-throughput analysis.

Further, in a method according to the present invention, the sequence information on a protein can be obtained with high likelihood. Hence, even though it is not registered in a database, a protein to be analyzed can be identified as a homologous protein by applying a homologous search to a database based on obtained sequence information thereon.

Furthermore, in a method according to the present invention, the internal sequence information of a protein can be obtained with high likelihood. Hence, there can be detected a difference between actually measured value and theoretical mass of a peptide, thereby enabling estimation of the presence or absence and a type, also a position of a modifying group not contained in genetic information.

Moreover, in a method according to the present invention, the internal sequence information of a protein can be obtained and besides the mass of a peptide fragment can be measured with high accuracy. Therefore, a method of identifying a protein utilizing these means in combination allows an identification ratio of protein identification and reliability of identification results thereof to increase drastically.

Incidentally, in a conventional analysis technique of C-terminal sequence, the limited cleavage using trypsin is substantially limited to cleavage at an arginine residue so that possibly it is difficult to perform sequence analysis in a protein with an arginine residue, which first emerges when viewed from the C-terminal side, being present at a position in the immediate proximity of the C-terminus or at a position far away from the C-terminus. On the other hand, in a method of the present invention, a used fragmentation technique of a protein does not suffer from the aforesaid limitation, i.e. enables the cleavage at a lysine residue. Thus in some cases, as to such problematic protein, its amino acid sequence can also be determined. If impossible, the present method make it possible to determine C-terminal amino acid sequence by employing fragmentation techniques other than that by trypsin.

### EXAMPLES

The present invention is described in more details below by means of an example. However, the scope of the present invention should not be understood as being restricted by such example.

### Example 1

In order to verify the utility of an analysis method of a protein concerning one embodiment of the present invention, first, a heme protein consisting of 153 amino acid residues, horse-derived myoglobin, is digested with trypsin, a plurality of resulting peptide fragments being recovered and dried to separate by liquid chromatography.

In the present Example, the globin peptide of horse myoglobin was used as an analyte, whose amino acid sequence is already known (SWISSPROT accession No. P68083, SEQ ID NO: 1). Using this peptide, the precision of identification for an amino acid sequence of each of peptide fragments identified by an analysis method according to the invention was verified. FIG. 1 shows a flow for steps of a method of analyzing a protein according to the present invention.

(Preparation of Peptide Fragments by Tryptic Digestion)

Two μg of horse myoglobin was subjected to 12.5% polyacrylamide gel electrophoresis, then a target band of a globin peptide chain was identified by Coomassie Brilliant Blue staining. This band was cut out and a trypsin-containing aqueous solution was added to a vial in which the gel slice was fed to conduct fragmentation of the peptide chain in a state that the chain was held by the gel carrier. In the above-stated trypsin-containing aqueous solution, trypsin was present at a concentration of 0.067 μg/μl in an ammonium bicarbonate buffer (pH 8). In the tryptic digestion process, the enzymatic reaction was performed at 37° C. for 4 hours under stirring. A plurality of peptide fragments produced by cleavage at the C-terminal side of lysine or arginine residue by the tryptic digestion become so easy to elute from the gel carrier as to elute in the trypsin solution contained in the vial. After the tryptic digestion treatment step was completed, the fragmentated peptides eluting from the gel in the trypsin solution contained in the vial were recovered and dried.

(Fractionation by Liquid Chromatography)

A sample containing a plurality of the peptide fragments recovered and dried by the aforementioned method was dissolved in an aqueous solution containing 35 μl of 0.1% trifluoroacetic acid (TFA), 2% acetonitrile, from which 4 μl and 20 μl were aliquoted to subject each aliquot to separate by liquid chromatography. The separation conditions are as follows:
HPLC system: MAGIC 2002 (Michrom BioResources, inc.)
Column: MAGIC C18 (0.2×50 mm, Michrom BioResources, inc.)
Elute A: 0.1% formic acid, 2% acetonitrile aqueous solution
Elute B: 0.1% formic acid, 90% acetonitrile aqueous solution
Gradient: a linear increase of the concentration of Elute B from 5% to 65% for 30 min
Flow rate: 70 μl/min
Flow rate through column: ca. 7 μl/min (under control by splitter)
   (Operation for Successive Release Reaction of C-terminal Amino Acids on a Plate)

Peptide fragments contained in a fraction of the above sample of 20 μl separated by LC was dropped and dried onto a plate for mass spectrometric measurement to conduct successive release reaction of C-terminal amino acids as shown below. Meanwhile, a peptide fragment contained in a fraction of the above sample of 4 μl separated by LC was dropped and dried onto a plate for mass spectrometric measurement to measure by means of a mass spectrometer without conducting successive release reaction of C-terminal amino acids.

Operations of pretreatment by acetylation, releasing reaction of C-terminal amino acids, and post-treatment by hydration were all conducted inside a desiccator. Specifically, on the bottom of the desiccator was placed a dish, the following reagents each of 5 ml being fed therein to arrange an internal dish on which the plate spotted with the above sample was placed. The desiccator inside was evacuated by means of a vacuum pump for 1 min to reduced pressure condition and then sealed in an airtight condition by turning off a cock. The desiccator under this airtight condition was kept warm at the following temperatures for their respective time intervals in sequence to allow the reagents of vaporous form supplied from the liquid-stated reagents in the container to contact (act on) the dry sample on the plate.

(1) Pretreatment of acetylation: using acetic anhydride with 5% volume of acetic acid added thereto as a reagent, reaction was carried out at room temperature overnight.

(2) Releasing reaction of C-terminal amino acids: using acetic anhydride with 5% volume of trifluoroacetic acid (TFA) added thereto as a reagent, reaction was carried out at 40° C. for 4 hours.

(3) Post-treatment operation of hydration: using aqueous solution with 20% volume of DMAE dissolved therein, hydration was carried out at 60° C. for 2 hours.

After each reaction, there developed an extremely reduced pressure condition. So, after each reaction was completed, the desiccator inside was once returned to normal pressure by introducing argon gass, then open its lid, reagents being exchanged.

(Mass Spectrometry Measurement)

Both the sample being subjected to successive release of C-terminal amino acids and the sample being not subjected to such reaction underwent mass spectrometry with use of MALDI-TOF-MS system (Voyager, Applied Biosystems, inc.).

The results are shown in FIGS. 3-5. FIG. 3 shows mass spectrum measured without carrying successive release reaction of C-terminal amino acids with respect to a sample originating from one fraction separated by LC. In general, it is difficult to isolate one peptide by LC. So it is found out that a plurality of components (peptide fragments) are mixed in mass spectrum shown in FIG. 3. Meanwhile, FIG. 4 indicates mass spectrum measured with carrying successive release reaction of C-terminal amino acids with respect to a sample originating from the same fraction. Therefrom, it is found out that by the reaction, there exist a plurality of peaks caused by cleavage of an original peptide fragment.

The result of analysis of an amino acid sequence by means of mass spectra of these samples is described below.

(Analysis of Mass Spectrum)

Analysis of mass spectrum obtained by the aforementioned operations is explained below. FIG. 2 is a flow chart of steps showing the analysis flow.

[Step (a)] Selection of a Peak Serving as a Reference (a Starting Point) for Analysis

(1) In the mass spectrum of an original peptide fragments shown in FIG. 3, the peak group including three or more isotopic peaks, which have intensities of not less than 5% of an intensity of maximum peak is sorted to select only one peak derived from a single isotope.

(2) Assuming that the peak selected in (1) originates from the peptide fragment, mass numbers of acetylated forms thereof are calculated. There is a possibility that by addition of an acetyl group, an N-terminal amino group is acetylated, and besides the e-amino group on a lysine residue contained in a peptide chain undergoes N-acetylation, and further the hydroxyl groups present in a serine or threonine residue and the phenolic hydroxyl group of a tyrosine residue are subjected to O-acetylation. Therefore, it is required to take into consideration at least three kinds of acetylated forms: monoacetylated form, diacetylated form and triacetylated form.

[Step (b)] Identification of a Peak Serving as a Reference (a Starting Point) for Analysis

(3) In the mass spectrum, shown in FIG. 4, in the case of performing successive release reaction of C-terminal amino acids, it is searched whether the peaks each selected or calculated in (1) and (2) are present or not. If these peaks are present, the analysis proceeds to the next step. However, if these peaks are absent, the analysis returns to (1) to select a new peak.

(4) Values are calculated by subtracting the mass differences caused by removal of an arginine residue and an acetyllysine residue (156.10 Da and 170.11 Da, respectively) as well as, in addition to these, the mass differences in the case of involving excessive dehydration (174.11 Da and 188.12 Da, respectively), from the mass of the peaks searched in (3).

[Step (c)] Identification of a Peak Concerning Removal of Only One C-terminal Amino Acid

(5) In the mass spectrum shown in FIG. 4, the single isotopic peaks having identical mass numbers to those of calculated in (4) is present. Here, the presence of these peaks indicates the presence of a candidate peptide with one amino acid released from the C-terminus, resulting in continuing to the next step (6). However, when not present, there is a possibility that it is a candidate peptide without arginine or lysine being present at the C-terminus, i.e. a C-terminal peptide, resulting in continuing to the next step (7).

[Step (d)] Determination of an Amino Acid Sequence of a Peptide Originating from an Internal Portion of a Protein

(6) Relative to the peak searched in (5) as mentioned above to be used as a reference (a starting point), there is identified a peak group corresponding to a series of reaction products which have been sequentially truncated by one amino acid from the C-terminus, and based on the peak group, there is calculated decreases in molecular weight caused by successive release of amino acids from C-terminus, and then based on the molecular weight decreases, there is identified a series of amino acid sequences released in sequence.

(7) In cases where by the search in (5) above, a peak candidate of a peptide of interest is not obtained, it is estimated that the C-terminus is neither lysine nor arginine. In such case, for the spectrum from which there are excluded the peaks having been chosen in (1)-(6) as each stated above, there is again conducted identification of a peak group corresponding to a series of reaction products which have been sequentially truncated by one amino acid from the C-terminus, and based on the peak group, there is calculated decreases in molecular weight caused by successive release of amino acids from C-terminus, and then based on the molecular weight decreases, there is identified a series of amino acid sequences released in sequence, which enables analysis of C-terminal amino acid sequence of a protein as an analyte.

The above-stated procedure makes it possible to identify a peak pair corresponding to releasing reaction of C-terminal amino acids of a peptide fragment, and this analysis procedure can be employed as a reference (a starting point) for spectrum analysis of a next amino acid residue. This technique is useful also in cases where a plurality of components are mixed so that also in such case, highly reliable sequencing can be implemented.

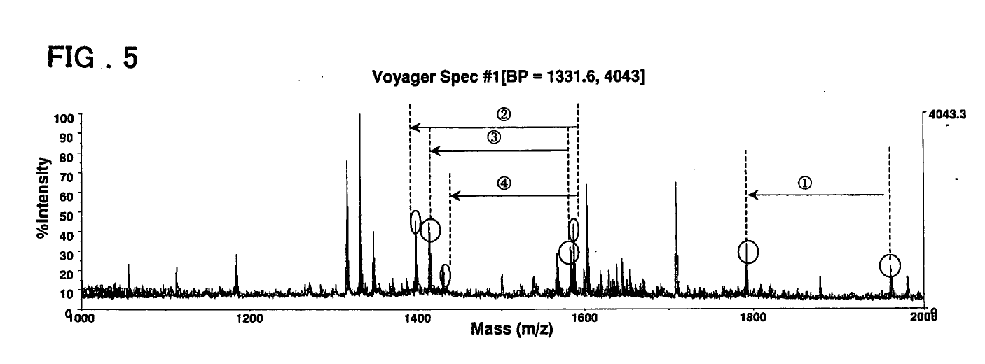

FIG. 5 shows an enlarged view of a region of m/z=1,000˜2,000 in order to represent the result of the spectrum analysis shown in Fif. 4 more specifically. Arrows indicated by the numbers 1 to 4 in the diagram show peaks found out in the search of (5) relative to the peaks searched in (3) to be each used as a reference. 1 and 2 both denote peak pairs corresponding to decreases in molecular weight by degrading acetyllisine involving excessive dehydration. 3 and 4 represent peak pairs corresponding to decreases in molecular weight caused by degradation of arginine involving excessive dehydration and by degradation of arginine, respectively. Thus it is predicted that the fraction analyzed contains a mixture of a plurality of peptides. As a result of more detailed analysis, it is found that the peaks present at the endpoints of 3 and 4 are peaks of oxidized forms of adjacent peaks, thus these candidates being excluded. Therefore, in the present example, the presence of a mixture of two kinds of peptides 1 and 2 has been revealed.

As a result of analyzing a plurality of fractions by means of the above-described method, sequence information shown in Table 1 below has been acquired.

TABLE 1
Result of analysis of amino acid sequence of myoglobin

| | | | | |
|---|---|---|---|---|
| | | m/z of | | |
| | | original | m/z of | |
| | | peptide | reaction | |
| Assignment | Amino acid sequence | fragment | product | Remarks |

| | | | | |
|---|---|---|---|---|
| [119-133] | [HPGNFGADAQ]GAMTK | 1502.7 | | |
| 2Ac[119-133][HPGNFGADAQ]GAMTK | | 1586.8 | | |
| Ac[119-132]-18 | [HPGNFGADAQ]GAMT | | 1397.7 | |
| Ac[119-131] | [HPGNFGADAQ]GAM | | 1314.6 | |
| Ac[119-130][HPGNFGADAQ]GA | | 1183.6 | | |
| Ac[119-129][HPGNFGADAQ]G | | 1112.6 | | |
| Ac[119-128][HPGNFGADAQ] | | 1055.5 | | |
| [80-96] | [GHHEAELKPLAQ]SHATK | 1854 | | |
| 3Ac[80-96] | [GHHEAELKPLAQ]SHATK | | 1980.1 | |
| 2Ac[80-95]-18 | [GHHEAELKPLAQ]SHAT | | 1791 | |
| 2Ac[80-94] | [GHHEAELKPLAQ]SHA | | 1708.9 | |
| 2Ac[80-93] | [GHHEAELKPLAQ]SH | | 1637.9 | |
| 2Ac[80-92] | [GHHEAELKPLAQ]S | | 1500.8 | |
| 2Ac[80-91] | [GHHEAELKPLAQ] | | 1412.7 | |
| [32-42] | [LFTGHPETL]EK | 1271.6 | | |
| 2Ac[32-42] | [LFTGHPETL]EK | | 1355.7 | -18 peak was bigger, |
| | | | | 3Ac was also observed |
| Ac[32-41] | [LFTGHPETL]E | | 1185.6 | |
| Ac[32-40] | [LFTGHPETL] | | 1056.6 | |
| [17-31] | [VEADIAGHGQ]EVLIR | 1606.8 | | |
| Ac[17-31] | [VEADIAGHGQ]EVLIR | | 1648.8 | |
| Ac[17-30] | [VEADIAGHGQ]EVLI | | 1492.7 | |
| Ac[17-29] | [VEADIAGHGQ]EVL | | 1379.6 | |
| Ac[17-28] | [VEADIAGHGQ]EV | | 1266.9 | |
| Ac[17-27] | [VEADIAGHGQ]E | | 1166.5 | trace, -1 mass shift |
| Ac[17-26] | [VEADIAGHGQ] | | 1038.4 | Trace |
| [64-77] | [HGTVVLTALGG]ILK | 1378.9 | | |
| 2Ac[64-77]+Na | [HGTVVLTALGG]ILK | | 1484.8 | |
| 2Ac[64-76]+Na | [HGTVVLTALGG]IL | | 1313.7 | |
| 2Ac[64-75]+Na | [HGTVVLTALGG]I | | 1200.6 | |
| 2Ac[64-74] | [HGTVVLTALGG] | | 1066 | |

In the Table above, the column “m/z of the original peptide fragment” shows mass to charge ratio corresponding to a peptide not subjected to releasing reaction of C-terminal amino acids chosen in step (a) of mass spectrum analysis procedure, and “m/z of reaction products” shows mass to charge ratio of detected truncated products of C-terminal amino acids. As evident from these results, partial sequences of five peptide fragments have been able to be determined.

It should be noted that other objects, features and aspects of the present invention will become apparent in the entire disclosure and that modifications may be done without departing the gist and scope of the present invention as disclosed herein and claimed as appended herewith.

Also it should be noted that any combination of the disclosed and/or claimed elements, matters and/or items may fall under the modifications aforementioned.

[Sequence Listing]

What is claimed is:

1. A method of analyzing a protein comprising the steps of:
(A) cleaving a protein to be analyzed limitedly at a specified amino acid position to prepare a mixture of plural peptide fragments;
(B) isolating and purifying one or more peptide fragments from said peptide fragment mixture;
(C) releasing successively C-terminal amino acids of said isolated and purified peptide fragment by chemical reactions to prepare a mixture containing a series of resulting products;
(D) subjecting individually to mass spectrometry both of said successively released reaction products and unreacted peptide fragments not subjected to said successive release reaction; and
(E) analyzing results of said mass spectrometry to obtain chemical structure information of the protein to be analyzed.

2. The analysis method of claim 1 wherein
said isolating and purifying step of step (B) comprises fractionating a plurality of fractions by liquid chromatography.

3. The analysis method of claim 1 wherein
step (B) comprises a step of dividing said peptide fragment mixture into two fractions, and a step of, with respect to each of said fractions, isolating and purifying one or more peptide fragments; and
step (C) comprises a step of releasing successively the C-terminal amino acids from the peptide fragment resulting from the step of isolating and purifying one of said divided fractions to prepare a mixture containing a series of resulting products thereof, and a step of maintaining a peptide fragment resulting from the step of isolating and purifying the other fraction without subjecting it to said successive release reaction to prepare unreacted peptide fragments.

4. The analysis method of claim 1 wherein
said chemical reaction in step (C) is conducted on a plate for mass spectrometric measurement.

5. The analysis method of claim 4 wherein
step (C) comprises at least the following steps of:
a pretreatment step of allowing an alkanoic acid anhydride and alkanoic acid both of vapor or droplet phase, which are supplied from a mixture of the alkanoic acid anhydride with a small amount of the alkanoic acid added thereto, to contact with a dry sample on the plate in a dry atmosphere at a temperature selected in a range of 10 to 60° C., thereby protecting the N-terminal amino group of the peptide fragments as well as the amino group on the side chain of the lysine residue which may be included in the peptides by means of N-acylation;
allowing, on the plate, an alkanoic acid anhydride and perfluoroalkanoic acid both of vapor or droplet phase, which are supplied from a mixture of the alkanoic acid anhydride with a small amount of perfluoroalkanoic acid added thereto, to contact with the dry peptide sample after N-acylation protection in a dry atmosphere at a temperature selected in a range of 15 to 80° C., thereby releasing the C-terminal amino acids at the C-terminus of the peptide accompanied by cleavage of a 5-Oxazolone ring via formation of a 5-Oxazolone structure represented by the following general formula (III): 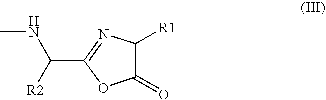
where R1 is a side chain of a C-terminal amino acid of the peptide, and R2 is a side chain of an amino acid residue positioned immediately before this C-terminal amino acid; and
a step of hydrolyzing the C-terminus of the peptide from the reaction products, which comprises applying, to a dried sample containing a series of reaction products obtained in said step of releasing the C-terminal amino acids successively on the plate, a post-treatment of removing the remaining alkanoic acid anhydride and perfluoroalkanoic acid, and then contacting post-treated peptides with a basic nitrogen-containing aromatic compound or a tertiary amine compound and water molecules, all of vapor or droplet phase, using an aqueous solution dissolving a basic nitrogen-containing, aromatic compound or a tertiary amine compound therein.

6. The analysis method of claim 5 wherein
step (C) comprises the following steps, which are repeated at least once:
adsorbing or depositing reaction reagents on the sample by setting the temperature of the plate for mass spectrometric measurement, on which the peptide fragments are spotted and dried, to lower than that of vapor of each reagent used in the steps of the reaction, and
volatilizing the reaction reagents by setting the temperature of the plate to higher than that of vapor of each reagent used in the steps of the reaction.

7. The analysis method of claim 5 wherein
said alkanoic acid anhydride is acetic anhydride.

8. The analysis method of claim 5 wherein
said perfluoroalkanoic acid has an acid dissociation constant (pKa) within a range of 0.3 to 2.5 and is a perfluoroalkanoic acid having 2 to 4 carbon atoms.

9. The analysis method of claim 5 wherein
a content ratio between said alkanoic acid anhydride and said perfluoroalkanoic acid both utilized in the step of releasing the C-terminal amino acids in association with the formation of the 5-Oxazolone structure and the cleavage of the 5-Oxazolone ring, is selected from a range of 1 to 20 volume of perfluoroalkanoic acid per 100 volume of alkanoic acid anhydride.

10. The analysis method of claim 5 wherein
in step (C), the direct deposition and volatilization of small droplets of reagents are repeated on the dried sample of the plate.

11. The analysis method of claim 5 wherein
in step (C), fresh reagent vapor is continually supplied onto the plate.

12. The analysis method of claim 11 wherein
in the step of releasing said C-terminal amino acids successively, fresh reagent vapor is continually supplied together with inert gas onto the plate.

13. The analysis method of claim 5 wherein
in step (C), a reagent is placed on the bottom of a desiccator, a plate is set on an internal dish inside the desiccator, and after evacuation and seal, the whole desiccator is heated to a predetermined temperature.

14. The analysis method of claim 1 wherein
step (E) comprises the following steps of:
comparing a mass spectrum of the reaction products obtained by said successive release of the C-terminal amino acids with a mass spectrum of unreacted peptide fragments not subjected to said successive release reaction;
identifying a series of amino acids successively released from the C-terminus of said peptide fragment by using the result of said comparison; and
arranging said identified amino acids to obtain amino acid sequence information of the protein.

15. The analysis method of claim 14 wherein
the step of identifying a series of the amino acids successively released from the C-terminus of said peptide fragment consists of:
(a) in the mass spectrum of said unreacted peptide fragment,
(a-1) sorting a peak group or groups including isotopic peaks of more than a given number, which have intensities greater than a given proportion of an intensity of maximum peak,
(a-2) selecting a single isotopic peak out of said peak group or groups,
(a-3) calculating mass of a peptide corresponding to said selected peak and one or more N-acylated forms of said peptide;
(b) in the mass spectrum of a series of said reaction products,
(b-1) searching the presence or absence of a peak which has the mass calculated in step (a-3),
(b-2) in case where said peak is present, choosing the peak as a candidate peak of the peptide fragment not truncated from the C-terminus or N-acylated form thereof,
(b-3) calculating a value by subtracting, from mass of the candidate peak of said N-acylated form, mass of the endmost amino acid residue of the peptide produced by said limited cleavage;
(c) in the mass spectrum of said a series of reaction products,
(c-1) searching the presence or absence of a peak which has the mass of the value calculated in step (b-3),
(c-2) in case where said peak is present, identifying the peak as a peak obtained by elimination of one C-terminal amino acid from said peptide fragment;
(d-1) identifying a peak group corresponding to a series of reaction products resulting from successive dissociation of C-terminal amino acids relative to said identified peak to be used as a reference, and based on said peak group, calculating decrease in molecular weight which is caused by successive release of amino acids from the C-terminus, and then
(d-2) identifying a series of amino acids resulting from successive release based on a series of calculated decrease in molecular weight.

16. The analysis method of claim 15 wherein
in the mass spectrum of a series of said reaction products, in case where a candidate peak of the peptide fragment not truncated from the C-terminus as identified in step (b-2), or the N-acylated form thereof is present, and a peak of the value calculated in step (b-3) is absent, it is judged that there is a possibility that the peptide fragment not truncated from the C-terminus is a C-terminal peptide fragment of a protein to be analyzed;
there is identified a peak group corresponding to a series of reaction products resulting from successive release of C-terminal amino acids relative to said judged peak to be used as a reference; based on said peak group, decrease in molecular weight is calculated which is caused by successive release of amino acids from C-terminus; and
based on a series of the calculated decrease in molecular weight, a sequence of successively released amino acids is identified to determine the identified amino acid sequence as a C-terminal amino acid sequence of a protein to be analyzed.

17. The analysis method according to claim 1 wherein, in case where genetic information of said protein to be analyzed is known, the information of chemical structure of the protein to be analyzed is obtained based on the mass spectrum of successively released reaction products of C-terminal amino acids without referring to the mass spectrum of unreacted peptide fragments.

18. The analysis method according to claim 1 wherein
in conducting mass spectrometry of said unreacted peptide fragment, a precise measurement is carried out with addition of an internal standard peptide.

19. The analysis method according to claim 1 wherein
said protein to be analyzed is limitedly cleaved at said specified amino acid position with a ptotease.

20. The analysis method according to claim 1 wherein
said mass spectrum is measured by MALDI-TOF-MS method.

21. The analysis method of claim 18, comprising the steps of:
choosing, based on mass of said unreacted peptide fragment obtained by the precise measurement with added internal standard peptide in conducting mass spectrometry of said unreacted peptide fragment, a protein candidate from which the peptide fragment of the measured precise mass can be derived, in reference to known protein database information;
determining, with respect to at least one peptide fragment subjected to said precise measurement, a part of C-terminal amino acid sequence thereof; and
identifying, among the protein candidates chosen by referring to said known protein database information, only a protein candidate having said amino acid sequence.

22. A method of identifying a homologous protein comprising the steps of:
determining a plurality of partial amino acid sequences of a protein to be analyzed using a method according to claim 1; and
implementing, using said a plurality of partial amino acid sequences determined, homology search for a protein registered in a protein database to identify a protein homologous to a protein to be analyzed.

23. A method of analyzing a posttranslational modification of a protein, the protein to be analyzed having a known amino acid sequence, said method comprising the steps of:
determining, using a method according to claim 1, mass numbers and partial amino acid sequences of a plurality of peptide fragments originating from the protein to be analyzed;
calculating, based on said known amino acid sequence, theoretical mass numbers of peptide fragments produced when the protein to be analyzed is subjected to a hypothetical limited cleavage at a specified amino acid position;
identifying the positions of said peptide fragments on the protein to be analyzed based on said partial amino acid sequences,
comparing, with respect to said peptide fragments with identified position, the mass measured by mass spectrometry with said theoretical mass;
judging, in case where there is a difference between said measured mass and said theoretical mass, that there is a possibility that a modifying group is present in said position-identified peptide fragment;
estimating, with respect to the peptide fragment judged that it has a possibility of the presence of said modification, a kind of said modifying group based on the difference between the theoretical mass and the measured mass; and
judging a position of an amino acid modified with said estimated modifying group from a position at which the difference disappears in the mass calculated from the spectrum measured in association with the successive release.

24. A method of analyzing a posttranslational modification of a protein, the protein to be analyzed having an unknown amino acid sequence, said method comprising the steps of:
identifying, using a method of claim 21, a gene corresponding to the protein to be analyzed;
predicting, based on the nucleotide sequence of said identified gene, an amino acid sequence of the protein to be analyzed;
calculating theoretical mass numbers of peptide fragments produced when the protein having said predicted amino acid sequence is subjected to a hypothetical limited cleavage at a specified amino acid position;
determining, using a method according to claim 1, mass numbers and partial amino acid sequences of a plurality of peptide fragments originating from the protein to be analyzed;
identifying the positions of said peptide fragments on the protein to be analyzed based on said partial amino acid sequences;
comparing, with respect to said peptide fragments with identified position, the mass measured by mass spectrometry with said theoretical mass;
judging, in case where there is a difference between said measured mass and said theoretical mass, that there is a possibility that a modifying group is present in said position-identified peptide fragment;
estimating, with respect to the peptide fragment judged that it has a possibility of the presence of said modification, a kind of said modifying group based on the difference between the theoretical mass and the measured mass; and
judging a position of an amino acid modified with said estimated modifying group from a position at which the difference disappears in the mass calculated from the spectrum measured in association with the successive release.

25. A method of determining a C-terminal amino acid sequence of a protein using chemical structure information obtained from the analysis of a protein sample by the method according to claim 1 wherein two or more limited cleavage techniques with site-specificity different from one another are applied, respectively,
the C-terminal amino acid sequence of a protein to be analyzed being determined by identifying a peptide fragment without C-terminal amino acid residue predicted from the used limited cleavage techniques.
